(19) United States
(12) Patent Application Publication
(10) Pub. No.: US 2006/0215114 A1
(43) Pub. Date: Sep. 28, 2006

(54) Local average curvature map for corneal topographers

(75) Inventor: Claus Flachenecker, Hayward, CA (US)

Correspondence Address: MICHAEL B. EINSCHLAG, ESQ., 25680 FERNHILL DRIVE, LOS ALTOS HILLS, CA, 94024, US

(73) Assignee: Carl Zeiss Meditec, Inc., Dublin, CA (US)

(21) Appl. No.: 11/091,890

(22) Filed: Mar. 28, 2005

(51) Int. Cl. A61B 3/00

(52) U.S. Cl. 351/246

(57) ABSTRACT

One embodiment of the present invention is a method for providing a local average curvature map of an eye that includes: (a) providing elevation data for points on a corneal surface of the eye; (b) selecting a set of elevation data in a neighborhood of each of a number of points on the corneal surface; (c) fitting a geometric figure at each of the number of points using the set of elevation data in each neighborhood; and (d) determining a curvature relating to the geometric figure at each of the number of points.

### TECHNICAL FIELD OF THE INVENTION

One or more embodiments of the present invention relate generally to visualization methods for corneal topographers, and in particular, a method for providing a local average curvature map for corneal topographers.

### BACKGROUND OF THE INVENTION

Corneal topography is a process for mapping surface curvature of a cornea similar to that for making a contour map of land. As is well known, the cornea is a clear membrane that covers the front of the eye and is responsible for about 70 percent of the eye's focusing power. To a large extent, the shape of the cornea determines the visual ability of an otherwise healthy eye—a perfect eye has an evenly rounded cornea, but if the cornea is too flat, too steep, or unevenly curved, less than perfect vision results. The purpose of corneal topography is to produce a detailed description of the shape and power of the cornea.

Of all the technologies currently available, corneal topography provides the most detailed information about the curvature of the cornea—thousands of measurements are taken and analyzed rapidly to provide a “3-D” color map perspective of the cornea's shape. The information provided by the color map is useful in evaluating and correcting astigmatism (i.e., measuring astigmatism is important for planning refractive surgery, fitting contact lenses, and calculating intraocular lens power), in monitoring corneal disease, and in detecting irregularities in the corneal shape. The corneal map is interpreted much like other topography maps, i.e., cool shades of blue and green represent flatter areas of the cornea,. while warmer shades of orange and red represent steeper areas.

Features of the cornea are traditionally determined by evaluating axial curvature (sometimes also referred to as sagittal curvature) and tangential curvature (sometimes also referred to as instantaneous curvature or meridional curvature) maps that are calculated/reconstructed by corneal topography systems. Axial and tangential maps ought to be displayed in units of radii of curvature (i.e., in mm) at each corneal surface point, however, for ophthalmologists who better understand more clinically used units of diopters (D) in ophthalmic practice, typical corneal topographers display curvature in units of keratometric D (as is well known, a keratometric diopter=337.5/radius of curvature). As is well known, the use of keratometric diopter is a simplification that ignores the fact that the refracting surface is an air-tear interface, and does not account for oblique incidence of incoming light in the corneal periphery. As a result, it “miscalculates” a true corneal refractive index of 1.376 to 1.3375 to correct for some of these factors—that is why these diopters more correctly are termed keratometric diopters to distinguish them from diopters expressing more precisely the true refractive power at a certain corneal point.

The above-described curvature maps have one feature in common, i.e., to provide them, the cornea is sliced into meridians that originate from a vertex of the cornea, and the curvature is calculated based on this directional slice—this method is the same as calculating a “directional curvature” along the meridian. As is known, such calculation and subsequent visualization of these directional curvatures is a tool that is useful in depicting astigmatism and eccentricity of an eye. See for example, an article by C. Campbell entitled “Reconstruction of the Corneal Shape with the MasterVue Corneal Topography System” in Optometry and Vision Science, Vol. 74, No. 11, November 1997, pp. 899-905 and an article by M. C. Corbett entitled “Corneal topography Basic principles and applications to refractive surgery” in Optometry Today, Feb. 25, 2000, pp. 33-41, both of which articles are incorporated herein by reference.

However, a main disadvantage in using such maps lies in their dependence on the position of the vertex of the cornea. For example, if a patient looks just slightly sideways during the measurement, the vertex or origin of the meridional coordinate system will shift, and the curvatures will change, even though the shape of the cornea is unchanged.

In addition to the above-described axial and tangential curvature maps, a corneal topographer may provide an elevation map. For example, in one such elevation map, the corneal topographer displays the elevation of a point on the corneal surface (i.e., a height of the point on the corneal surface relative to a reference surface, for example, a mathematical approximation of the actual corneal surface called a best-fit sphere is calculated by software for every elevation map separately). Other visualizations may provide corneal elevation data in comparison to a best-fit ellipsoid or toroid. As is well known, the map of elevation difference between the reference surface/elevation and the reconstructed corneal surface/elevation reveals corneal irregularities, and such elevation maps have an advantage of being independent of vertex position. However, they suffer from a disadvantage of displaying elevation differences relative to the reference surface in microns, and not in curvature values that a doctor is used to seeing and interpreting.

In light of the above, there is a need to develop visualization maps that overcome the above-described disadvantages.

### SUMMARY OF THE INVENTION

One or more embodiments of the present invention satisfy one or more of the above-identified needs in the art. In particular, one embodiment of the present invention is a method for providing a local average curvature map of an eye that comprises: (a) providing elevation data for points on a corneal surface of the eye; (b) selecting a set of elevation data in a neighborhood of each of a number of points on the corneal surface; (c) fitting a geometric figure at each of the number of points using the set of elevation data in each neighborhood; and (d) determining a curvature relating to the geometric figure at each of the number of points.

### BRIEF DESCRIPTION OF THE DRAWING

FIG. 1 shows an axial curvature map of an eye with suspected pellucid marginal corneal degeneration that was provided by a corneal topographer in accordance with prior art techniques; and

FIG. 2 shows a local average curvature map of the eye that was provided in accordance with one or more embodiments of the present invention using elevation data provided by the corneal topographer.

### DETAILED DESCRIPTION

In accordance with one or more embodiments of the present invention, a local average curvature map (LACM) is calculated from data provided by a corneal topographer that overcomes the disadvantages of the prior art discussed in the Background of the Invention. In particular, and in accordance with one or more embodiments of the present invention, elevation data provided by the corneal topographer are utilized to provide the LACM. As will be described in detail below, two main features of an LACM are: (a) independence of vertex position (or coordinate system origin) of the cornea, and (b) suppression of effects apparent in the map due to corneal astigmatism. As will be described in detail below, since the values of an LACM are calculated by obtaining an average of curvatures (whereas the axial/tangential curvature maps provided by prior art techniques display directional curvatures), effects apparent in the map due to corneal astigmatism are suppressed in the LACM. As one can readily appreciate, suppression of effects apparent in the map due to corneal astigmatism is a great advantage for a doctor, because it enables the doctor to readily distinguish pathological corneal anomalies from. “harmless” corneal astigmatism.

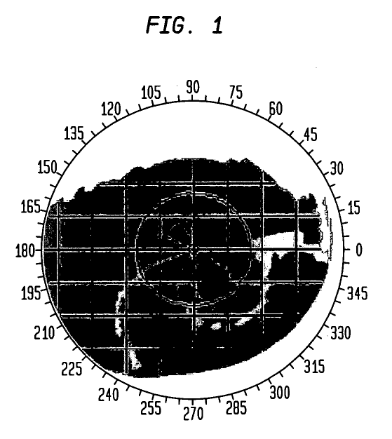

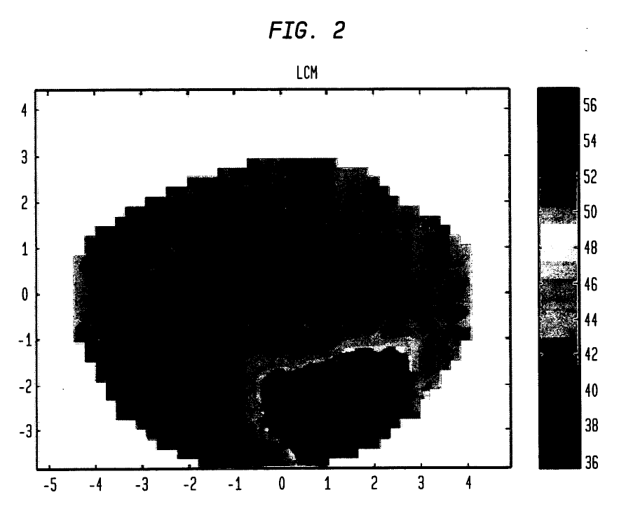

The advantages of the LACM map can be readily appreciated by referring to FIGS. 1 and 2. FIG. 1 shows an axial curvature map of an eye with suspected pellucid marginal corneal degeneration that was provided by a corneal topographer in accordance with prior art techniques. FIG. 2 shows a local average curvature map (LACM) of the eye that was provided in accordance with one or more embodiments of the present invention using elevation data provided by the corneal topographer. As one can readily appreciate, the effects apparent in the map of FIG. 1 that are due to corneal astigmatism (i.e., the bow-tie in the center of the axial curvature map shown in FIG. 1) have been suppressed or eliminated in FIG. 2 (i.e., the flat surface of the LACM shown in FIG. 2). As a result, and advatageously in accordance with one or more embodiments of the present invention, a corneal “bulge” with higher average curvature than the surrounding tissue is exposed in FIG. 2 to enable one to identify the pellucid marginal corneal degeneration. This advantage is readily seen when the axial curvature map of FIG. 1 is compared with the LACM of FIG. 2.

An LACM is provided in accordance with one or more embodiments of the present invention as follows. First, a corneal topographer measures a corneal surface in accordance with any one of a number of methods that are well known to those of ordinary skill in the art, for example, see the article by C. Campbell entitled “Reconstruction of the Corneal Shape with the MasterVue Corneal Topography System” in Optometry and Vision Science, Vol. 74, No. 11, November 1997, pp. 899-905 to provide elevation data (x_{e,i},y_{e,i,}z_{e,i}) over the surface of the cornea—the data are preferably defined in a cartesian coordinate system in the corneal plane (as is well known such a corneal plane touches the corneal vertex, with positive values of x extending from nose to ear, positive values of y extending from eye to forehead, and positive values of z extending toward the corneal surface). Then, for each data point of the LACM, the corneal elevation data within a predetermined neighborhood of the data point is extracted from the dataset, and the local average curvature is determined by calculating, for example and without limitation, a best-fit sphere for the subset. The curvature for a suitable number of points is then displayed as the LACM, for example and without limitation, in color-coded format in accordance with any one of a number of methods that are well known to those of ordinary skill in art. Note that, in accordance with one or more embodiments of the present invention, elevation data need not exist at a point of interest, i.e., a point at which a local average curvature will be determined. In other words, the points selected at which a local average curvature will be determined need not be points for which corneal elevation data was provided.

In particular, the following describes mathematical steps used to calculate the LACM in accordance with one or more embodiments of the present invention. As discussed above, the LACM is of best use if it is defined in a cartesian coordinate system in the corneal plane, as a function of x and y, a local distance metric delta (Δ), and reconstructed corneal elevation data over the corneal surface provided by the corneal topographer and expressed as a “cloud” of n surface points in 3D space (x_{e,1},y_{e,1},z_{e,1})^{T} . . . (x_{e,n},y_{e,n},z_{e,n})^{T}. The data set selected by local distance metric Δ ought to be large enough to provide low noise for the LACM (also note that if local distance metric Δ is too small, the LACM will approach the local data set). Appropriate values of α may be determined routinely and without undue experimentation using these criteria. Thus, the LACM may be defined as follows:

$$LACM ( x , y ) = f ( x , y , Δ , ( \begin{matrix}{x}_{e , 1} \\ {y}_{e , 1} \\ {z}_{e , 1}\end{matrix} ) , ( \begin{matrix}{x}_{e , 2} \\ {y}_{e , 2} \\ {z}_{e , 2}\end{matrix} ) , … , ( \begin{matrix}{x}_{e , n} \\ {y}_{e , n} \\ {z}_{e , n}\end{matrix} ) ) \qquad ( 1 )$$

The first step in the calculation is to determine elevation data points that lie within a neighborhood around a point of interest (x,y) (in accordance with one or more embodiment of the present invention, elevation data need not exist at the point of interest (x,y)). This can be done in a variety of ways, but in accordance with one or more embodiments of the present invention, one may utilize a point-distance metric Δ in the x/y-plane between (x,y) and each elevation data point as a filter:

Use

$$( \begin{matrix}{x}_{e , i} \\ {y}_{e , i} \\ {z}_{e , i}\end{matrix} )$$

for later operations, only if (x_{e,i}−x)²+(y_{e,i}−y)²≦Δ² (2)

This generates a “filtered” cloud of m corneal surface points in 3D space (x₁,y₁,z₁)^{T} . . . (xₘ,yₘ,zₘ)^{T} in a neighborhood around (x,y).

Now the LACM can be calculated in a variety of different ways, for example and without limitation, a fit to a sphere, an ellipsoid, a torus, and so forth. The following illustrates an embodiment of the present invention which entails least-square fitting a sphere against the filtered out elevation data points in a local neighborhood.

A sphere is generally defined by the equation:
(x−x₀)²+(y−y₀)²+(z−z₀)²=R² (3)
which is equivalent to:
x²+y²+z²−2x₀x−2y₀y−2z₀z+(x₀²+y₀²+z₀²−R²)=0 (4)

Given the filtered set of elevation data points (x₁,y₁,z₁)^{T} . . . (xₘ,yₘ,zₘ)^{T}, the following matrix representation is equivalent to equation (4) for each single data point:

$$( \begin{matrix}{x}_{1} & {y}_{1} & {z}_{1} & 1 \\ {x}_{2} & {y}_{2} & {z}_{2} & 1 \\ ⋮ & ⋮ & ⋮ & ⋮ \\ {x}_{m} & {y}_{m} & {z}_{m} & 1\end{matrix} ) ( \begin{matrix}a \\ b \\ c \\ d\end{matrix} ) = ( \begin{matrix}- {x}_{1}^{2} & - {y}_{1}^{2} & {z}_{1}^{2} \\ - {x}_{2}^{2} & - {y}_{2}^{2} & - {z}_{2}^{2} \\ & ⋮ & \\ - {x}_{m}^{2} & - {y}_{m}^{2} & - {z}_{m}^{2}\end{matrix} ) \qquad ( 5 )$$

where:
a=−2x₀
b=−2y₀
c=−2z₀
d=x₀²+y₀²+z₀²−R² (6)

Or, in a more compact representation:
Ak=u (7)
where:

$$A = ( \begin{matrix}{x}_{1} & {y}_{1} & {z}_{1} & 1 \\ {x}_{2} & {y}_{2} & {z}_{2} & 1 \\ ⋮ & ⋮ & ⋮ & ⋮ \\ {x}_{m} & {y}_{m} & {z}_{m} & 1\end{matrix} ) , u = ( \begin{matrix}- {x}_{1}^{2} & - {y}_{1}^{2} & {z}_{1}^{2} \\ - {x}_{2}^{2} & - {y}_{2}^{2} & - {z}_{2}^{2} \\ & ⋮ & \\ - {x}_{m}^{2} & - {y}_{m}^{2} & - {z}_{m}^{2}\end{matrix} ) and k = ( \begin{matrix}a \\ b \\ c \\ d\end{matrix} ) \qquad ( 8 )$$

As is well known to those of ordinary skill in the art, this over-determined set of equations can be “least-square” fitted by solving the following equation for k:
A^{T}A·k=A^{T}u (9)

This leads to:
k=(A^{T}A)⁻¹A^{T}u (10)

From the resulting vector k=(a b c d)^{T}, the local average curvature R can be extracted from eqn. (6) as follows:

$${R}_{( x , y )} = \frac{\sqrt{{a}^{2} + {b}^{2} + {c}^{2} - 4 d}}{2} \qquad ( 11 )$$

Finally, the local average curvature map (LACM) can either be expressed in “mm curvature radius” by:
LACM(x,y)=R_{(x,y)} (12)

or in “keratometric diopters” by:

$$LACM ( x , y ) = \frac{{n}_{ref} - 1}{{R}_{( x , y )}} \qquad ( 13 )$$

where n_{ref} is the corneal refractive index (usually taken as 1.3375).

This set of equations needs to be repeated for each location (x,y) of interest for which the LACM needs to be calculated, typically a predetermined number of points. Lastly, the LACM may be plotted using color codes in accordance with any one of a number of color-coding schemes that are well known to those of ordinary skill in the art. For example, one may utilize a color-coding scheme wherein cool shades of blue and green represent flatter areas of the cornea while warmer shades of orange and red represent steeper areas.

Although various embodiments that incorporate the teachings of the present invention have been shown and described in detail herein, those skilled in the art can readily devise many other varied embodiments that still incorporate these teachings.

What is claimed is:

1. A method for providing a local average curvature map of an eye that comprises:
providing elevation data for points on a corneal surface of the eye;
selecting a set of elevation data in a neighborhood of each of a number of points on the corneal surface;
fitting a geometric figure at each of the number of points using the set of elevation data in each neighborhood; and
determining a curvature relating to the geometric figure at each of the number of points.

2. The method of claim 1 which further comprises utilizing the curvatures to provide the local average curvature map in terms of curvature.

3. The method of claim 1 which further comprises utilzing the curvatures to provide the local average curvature map in terms of keratometric diopters.

4. The method of claim 2 which further comprises providing a color coded map of the local average curvature map.

5. The method of claim 3 which further comprises providing a color coded map of the local average curvature map.

6. The method of claim 1 wherein the geometric figure is a sphere.

7. The method of claim 6 wherein the step of determining comprises least square fitting elevation data in the neighborhood.

8. The method of claim 1 wherein the step of providing comprises using a corneal topographer to provide the elevation data.
